(19) United States
(12) Patent Application Publication
(10) Pub. No.: US 2024/0180589 A1
(43) Pub. Date: Jun. 6, 2024

(54) TWO-MODE MORCELLATION DEVICE

(71) Applicant: Gyrus ACMI, Inc. D/B/A Olympus Surgical Technologies America, Westborough, MA (US)

(72) Inventors: Charles A. Baker, Rogers, MN (US); Kester Julian Batchelor, Mound, MN (US); Theodore C. Blus, Arden Hills, MN (US); Joey Magno, Dudley, MA (US); Jake Terravecchia, Brighton, MA (US); Jeffrey D. Holton, Stanchfield, MN (US)

(21) Appl. No.: 18/365,502

(22) Filed: Aug. 4, 2023

Related U.S. Application Data
(60) Provisional application No. 63/429,590, filed on Dec. 2, 2022.

(51) Int. Cl. A61B 17/42; A61B 17/32

(52) U.S. Cl. CPC A61B 17/42; A61B 17/32; A61B 2017/320024; A61B 2017/4216; A61B 2217/005; A61B 2560/02; A61B 2560/04

(57) ABSTRACT

A medical device can include an elongate member that defines a lumen and a resection element that extends through the elongate member lumen. The elongate member can include a side window at a distal portion and a front window at a distal end of the elongate member and proximate to the side window. The front window can define an opening at the elongate member distal end. The resection element can be movable within the elongate member lumen where a distal end of the resection element can be exposed at the front window. The resection element can have a side window that is alignable with the elongate member side window. In addition, the resection element can define an electrically active electrode and the elongate member can define a return electrode.

### PRIORITY CLAIM

This application claims the benefit of priority to U.S. Provisional patent Application Ser. No. 63/429,590, filed Dec. 2, 2022, the contents of which are hereby incorporated by reference in their entirety.

### TECHNICAL FIELD

This document pertains generally, but not by way of limitation, to surgical devices that can be used for various surgical procedures. More specifically, but not by way of limitation, the present application relates to a surgical device that may be used to treat the reproductive system of a female patient.

### BACKGROUND

Growths can occur in the lining of a uterus. This can cause discomfort and interfere with menstruation and fertility if left untreated. The growths can include polyps and fibroids. Uterine polyps are growths that can form in the inner lining of a uterus. Uterine polyps can form when an overgrowth of endometrial tissue occurs. The polyp can attach to the endometrium and then extend into the uterus.

Fibroids are noncancerous growths that can develop in the wall of a uterus. Different types of fibroids include intramural, submucosal, subserosal, and pedunculated. Intramural fibroids can occur within the muscular walls of the uterus and can cause heavy bleeding. Submucosal fibroids can occur inside the uterine cavity or can abut the uterine cavity. Submucosal fibroids can also cause heavy bleeding. Subserosal fibroids can occur on the outer wall of the uterus and can cause bulk or pressure symptoms. Pedunculated fibroids can attach to the uterine wall by a stalk-like growth called a peduncle.

When a patient has a submucosal fibroid, a procedure can be used to examine the inside of the uterus and remove the fibroid via a resection. A morcellator can be inserted through the vagina and through the natural opening of the cervix. Typically, due to the diameter of the morcellator, the cervix must be dilated, which can be painful and require the patient to be under general anesthesia. As a result, an operating room is required and the recovery period due to the dilation can be increased.

Furthermore, current morcellators only include a blade that is disposed at a side of the hysteroscope, which can create a number of problems. First, having a blade disposed at a side of the morcellator limits the ability of the morcellator to resect tissue from a backwall of the uterus. Instead, a separate device must be used to resect tissue from the backwall, which increases the overall size of the morcellator since additional working instruments are required. Second, by virtue of only having a blade, coagulation cannot occur upon tissue resection until separate coagulation means are provided to the target site. Thus, a separate device must be used for coagulation, which again increases the overall size of the morcellator.

### SUMMARY

Accordingly, what is needed is a smaller diameter morcellator that allows for less dilation of the cervix and less anesthesia while at the same time allowing for tissue removal at a back wall of a target site and coagulation at the target site. This problem can be addressed by providing a two-mode morcellator having an elongate member that includes a front window at a distal tip. The elongate member can define a lumen through which a resection element can extend. In a first position, the resection element can extend from the front window in order to resect tissue. The elongate member can define a return electrode and the resection element can define an active electrode, which, in conjunction with the return electrode, can operate to resect tissue from a target site upon energization. During resection, the resection element can function to coagulate tissue at the target site during resection. The elongate member can also define a side window at a distal end thereof that is proximate the distal end and the front window. The resection element can define a side window that can align with the elongate member side window in a second position. The resection element side window can include a cutting edge that can function to resect tissue during reciprocation of the resection element within the elongate member.

The resection element can include a cross member that traverses an opening defined by the resection element. Either a portion of the cross member or the entire cross member can define an active element that can work in conjunction with the return element to resect tissue from a target site upon energization and rotation of the resection element.

The distal end of the elongate member can also include an endcap proximate the front window. The endcap can partially enclose the front window or fully enclose the front window. The end cap can be movably coupled with the elongate member between a closed position and an open position. In the open position, the resection element can extend from the front window to resect tissue at a target site.

A potential advantage includes the ability to reduce an amount of cervix dilation and patient anesthesia. More specifically, by providing a morcellator having the ability to resect tissue and coagulate tissue at a back wall of a uterus, an overall lateral size of the morcellator can be reduced, thereby reducing an amount by which the cervix should be dilated and potentially also reducing an amount of anesthesia necessary for a hysteroscopic procedure.

Another potential advantage relates to a device that can work in a small area by virtue of having a small size while having the ability remove large portions of tissue, such as large fibroids or large polyps.

### BRIEF DESCRIPTION OF FIGURES

FIG. 1 illustrates a morcellator having a resection assembly.

FIG. 2 shows a perspective view of a resection assembly.

FIG. 3 is a top view of the resection assembly of FIG. 2 in a first position.

FIG. 4 is a top view of the resection assembly of FIG. 2 in a second position.

FIGS. 5A and 5B show the resection of tissue from a target site.

FIGS. 6-11 are perspective views of alternative examples of a resection assembly.

FIG. 12 illustrates an alternative example of a resection assembly.

FIGS. 13 and 14 are side views of a resection assembly.

FIG. 15 is an alternative view of the resection assembly of FIG. 12.

FIG. 16 is an alternative example of a resection assembly.

FIG. 17 is a top view of the resection assembly of FIG. 16.

FIGS. 18 and 19 show alternative examples of a resection assembly.

FIG. 20 is a flowchart indicating a reprocessing method for the hysteroscope of FIG. 1.

### DETAILED DESCRIPTION

A morcellator can include an elongate member that includes a front window at a distal tip. The elongate member can define a lumen through which a resection element can extend. In a first position, the resection element can extend from the front window in order to resect tissue. The elongate member can define a return electrode and the resection element can define an active electrode, which, in conjunction with the return electrode, can operate to resect tissue from a target site upon energization. During resection, the resection element can function to coagulate tissue at the target site during resection. The elongate member can also define a side window at a distal end thereof that is proximate the distal end and the front window. The resection element can define a side window that can align with the elongate member side window in a second position. The resection element side window can include a cutting edge that can function to resect tissue during reciprocation of the resection element within the elongate member.

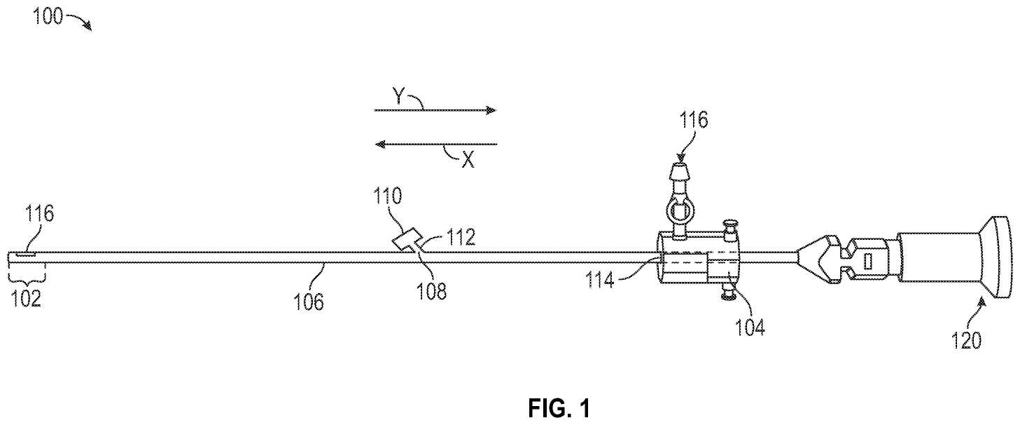

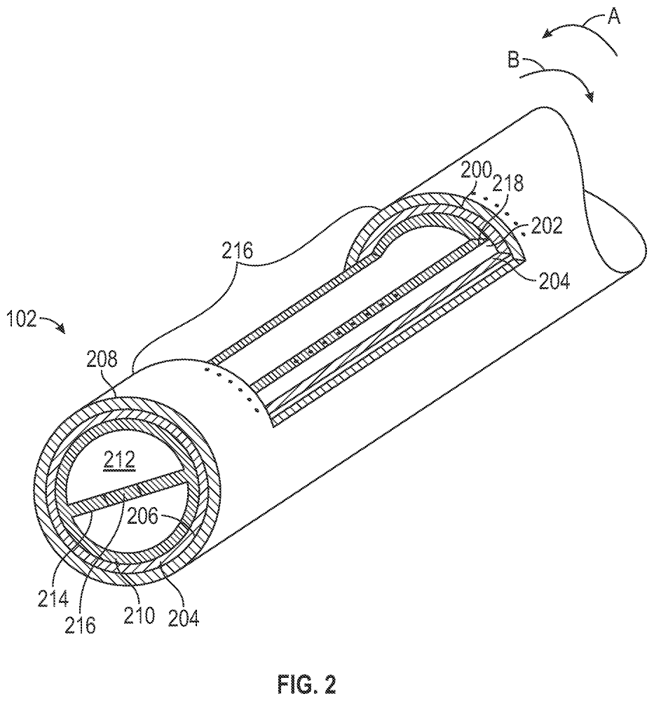

FIG. 1 shows an example of a morcellator 100 having a resection assembly 102. The morcellator 100 can include a housing 104, which can function to receive various components of the morcellator 100. Examples of such components can include, but are not limited to, an endoscope, a light source, an inflow channel, and an outflow channel. The morcellator 100 can also include an elongate member 106 extending from the housing 102. The elongate member 106 can include a lateral suction port 108 having a suction source connection 110. The suction source connection 110 can facilitate fluid coupling of the lateral suction port 108 with a suction source that can evacuate fluid from the lateral suction port 108. Thus, fluid and debris, such as material removed from a target site, can be removed from the target site and the morcellator 100 via the lateral suction port 108. In particular, fluid and debris can travel from the lateral suction port 108 along a suction port neck 112 to the suction source. While suction is described as occurring at the lateral suction port 108, suction can occur at different portions of the elongate member 106. For example, additionally or alternatively, the elongate member 106 can have a suction port located proximate a proximal end 114 of the elongate member 106.

The lateral suction port 108 can be used to remove debris resected from a target site during a resection procedure with the morcellator 100. The lateral suction port can also be used to remove irrigant provided to the elongate member 106 at an irrigation port 116 located at the elongate member proximal end 114. Irrigant can be provided to the elongate member from an irrigation source (not shown) that can be coupled to the irrigation port 116. The irrigant can be provided to a target site via the resection assembly 102.

The elongate member 106 can define a lumen 200 through which a resection element 202 can extend. In addition, an inner tube 204 can extend within the lumen 200 between the elongate member 106 and the resection element 200. The morcellator 100 can be a bipolar device where the resection element 202 can function as an active electrode and the elongate member 106 can function as a return electrode. The resection element 202 can be a conductor and extend from an electrical source (not shown) of the morcellator 100 to a distal end 118 (FIG. 1) of the elongate member 106. The elongate member 106 can also be a conductor and can be connected to ground. Examples of materials that can be used for the elongate member 106 and the resection element 202 can include stainless steel, aluminum, copper, or any other conductive material. The inner tube 204 can be an insulator disposed between the elongate member 106 and the resection element 202 when the morcellator 100 has a bipolar configuration. The inner tube 204 can be formed from any type of electrically insulative material, such as ceramic, any type of polymer, rubber, or the like.

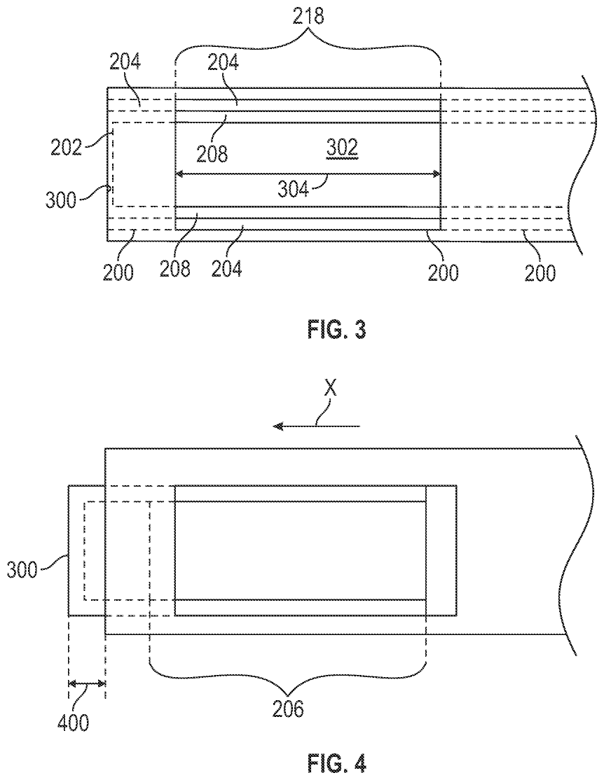

The elongate member 106 can have a front window 206 at a distal tip 208 where a distal end 210 of the resection element 202 is exposed at the elongate member front window 206. The resection element 202 can be slidable relative to the elongate member lumen 200 between a first position and a second position. In the first position, a surface 300 at the resection element distal end 210 can be flush with the elongate member distal tip 208, as shown in FIG. 3. The resection element 202 can be actuated along a direction X via a handle 120 (FIG. 1) to the second position shown in FIG. 4. In the second position, the resection element surface 300 protrudes from the elongate member front window 206 by a distance 400. The distance 400 may be in a range of 0.01 millimeters to about 5 millimeters. When the resection element 202 is in the second position of FIG. 4, the resection element surface 300 can be used to resect tissue at a target site.

The resection element 202 can define an opening 212 located at the elongate member distal tip 208 where a cross-member 214 can extend across the resection element opening 212. The resection element cross-member 214 and the resection element distal end 210 can form an active electrode in conjunction with the resection element 202. Moreover, in some examples, only the resection element cross-member 214 can form the active electrode where the resection element distal end 210 does not form part of the active electrode. Still further, only a portion 216 of the resection element cross-member 214 can form the active electrode of the resection element 202 instead of the entire cross-member 214. The resection element cross-member 214 can be formed from the same material as the resection element 202 as mentioned above.

When the resection element 202 is in the second position, the resection element distal end 210 and the cross-member 214 can protrude from the elongate member front window 206. In the second position, during tissue resection at a target site, such as resection of fibroids at a back wall of a uterus, the resection element distal end 210 and the cross-member 214 can be energized to form the active electrode and rotated along a direction A or a direction B. Rotation of the resection element cross-member 214 along one or both of the direction A or the direction B can enhance tissue resection at a back wall of a uterus. The resection element 202 may not include the cross-member 214. Here, the resection element distal end 210 can form the active electrode and resect tissue at a target site. Additionally, by virtue of using bipolar energy for resection, the resection element 202 and the resection element surface 300 can simultaneously coagulate a surface of the target site during tissue resection.

The elongate member 106 can include a side window 216 at the elongate member distal portion 118. The resection element 200 can include a side window 302 that is alignable with the elongate member side window 216. More specifically, the elongate member side window 216 and the resection element side window 302 can have a width 304 such that when the resection element 202 is in the first position in FIG. 3, the elongate member side window 216 and the resection element side window 302 align with each other.

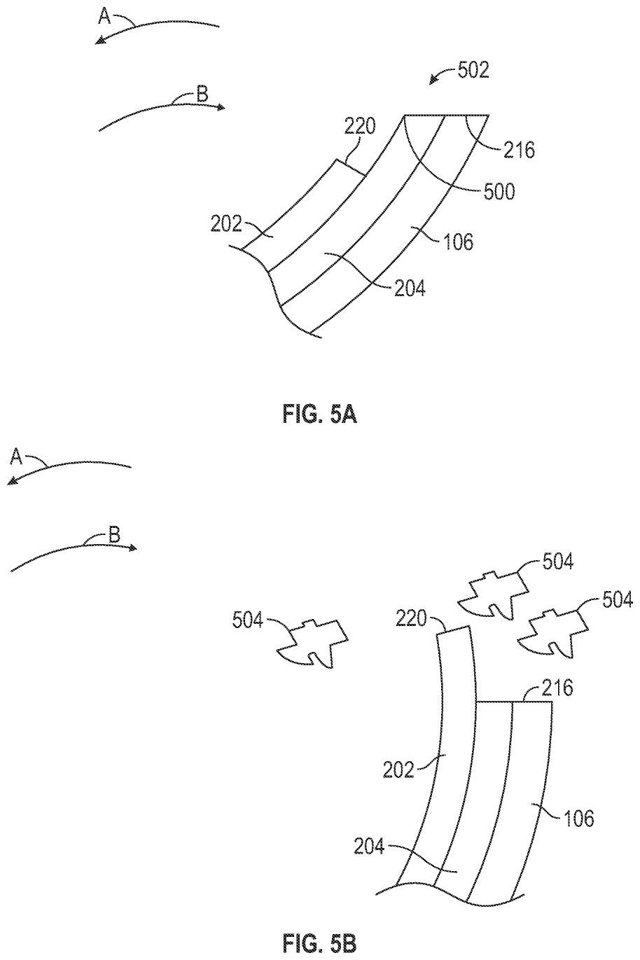

The resection element side window 302 can include a cutting edge 218, which can be used to resect tissue from a target site by virtue of being an active electrode. The cutting edge 218 can be a blade, a sharp edge, or the like. During use of the morcellator 100, tissue can be resected from a wall of a uterus via the elongate member side window 206. The resection element 202 can oscillate along the directions A and B within the elongate member 106 as shown in FIGS. 5A and 5B. The inner tube 204 can include an edge 500 that, in conjunction with the cutting edge 218, resects tissue from a target site 502. As the cutting edge 218 passes the inner tube edge 500 at the target site 502, debris 504, such as portions of fibroids, are resected from the target site 502, as shown in FIG. 5B. Suction provided at the suction port 108 and the suction 110 can facilitate removal of the debris 504 through a lumen provided by the resection element 202.

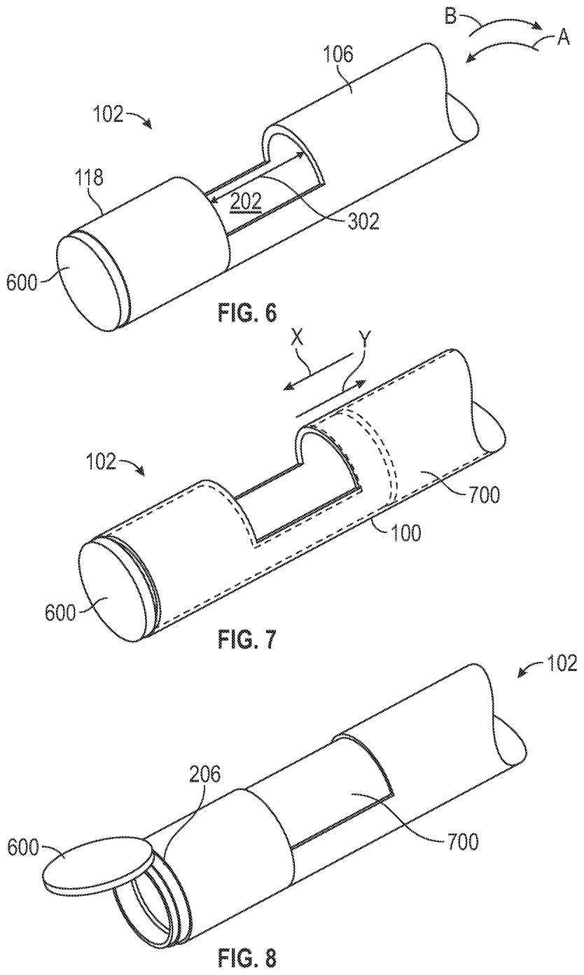

The resection assembly 102 can also have the configuration shown with reference to FIGS. 6-8. Here, the resection assembly 102 can include an end cap 600 located at the elongate member distal end 118 The end cap 600 can be movable between a closed position as shown in FIGS. 6 and 7 and an open position as shown in FIG. 8. The end cap 600 can be operatively coupled to the elongate member 106 via a hinge or any other type of mechanism that allows for movement of the endcap 600 between the closed and open positions. While an endcap is shown, any type of movable enclosure can be located at the elongate member distal end 118, such as a tricuspid valve, a yoke assembly, or the like. The morcellator 100 can include a sheath 700 that can be slidable relative to the elongate member 106 and the resection element 202 along the directions X and Y. The sheath 700 can be formed from the same material as the inner tube 204 and can function as an insulator when the elongate member 106 is a return electrode and the resection element 202 is an active electrode.

In a first position, the resection element 202 can have the configuration in FIG. 6, where the elongate member side window 218 (not shown) and the resection element side window 302 align with each other. In the first position, the sheath 700 can be retracted within the elongate member 106 as shown in FIG. 7, thereby exposing the elongate member side window 218 and the resection element side window 302. As such, in the first position, tissue resection at a target site can occur via the cutting edge 220, the elongate member side window 218, and the resection element side window 302, when the resection element 202 oscillates along the directions A and B. In the first position, the end cap 600 can be in the closed position and cover the resection element distal end 210. Accordingly, tissue resection only occurs via the cutting edge 220 at the elongate member side window 218 and the resection element side window 302.

In a second position, the sheath 700 can be moved along the direction X with the handle 120 into the configuration of FIG. 8. The sheath 700 can move the end cap 600 into an open position, thereby exposing the resection element distal end 210. In some examples, the end cap 600 can be formed of the same material as the elongate member 106 such that the end cap 600 can function as part of the return electrode defined by the elongate member 106, thereby allowing further extension of the resection element distal end 210 from the elongate member front window 206 in comparison to the example of FIG. 4. In the second position, the resection element distal end 210 can be used to resect tissue at a target site as discussed above. In addition, the sheath 700 can cover the elongate member side window 218 and the resection element side window 302. Thus, the cutting edge 220 is not used to resect tissue in the second position.

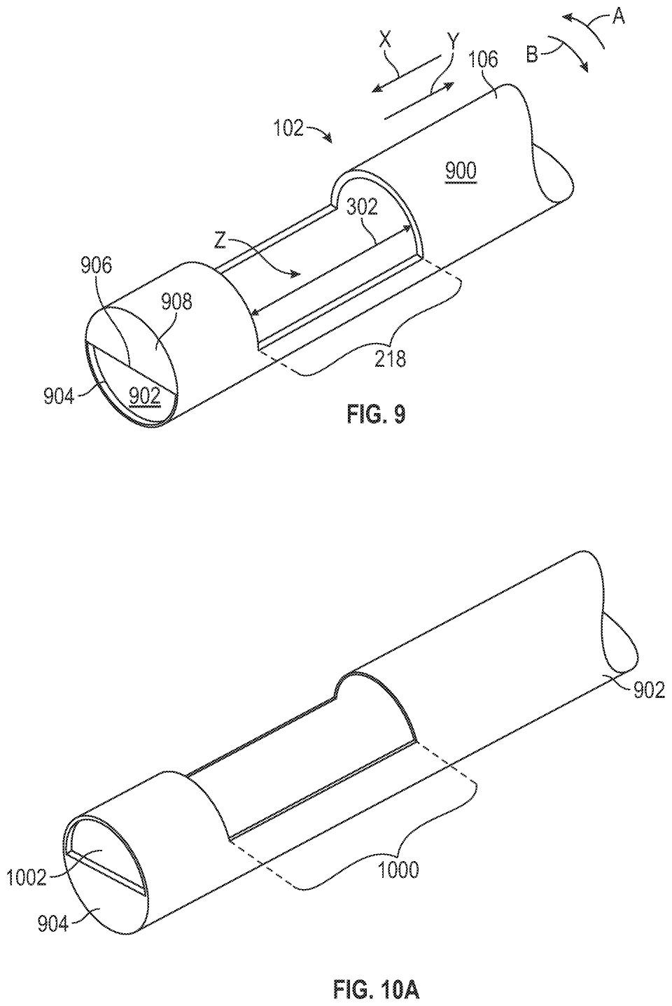

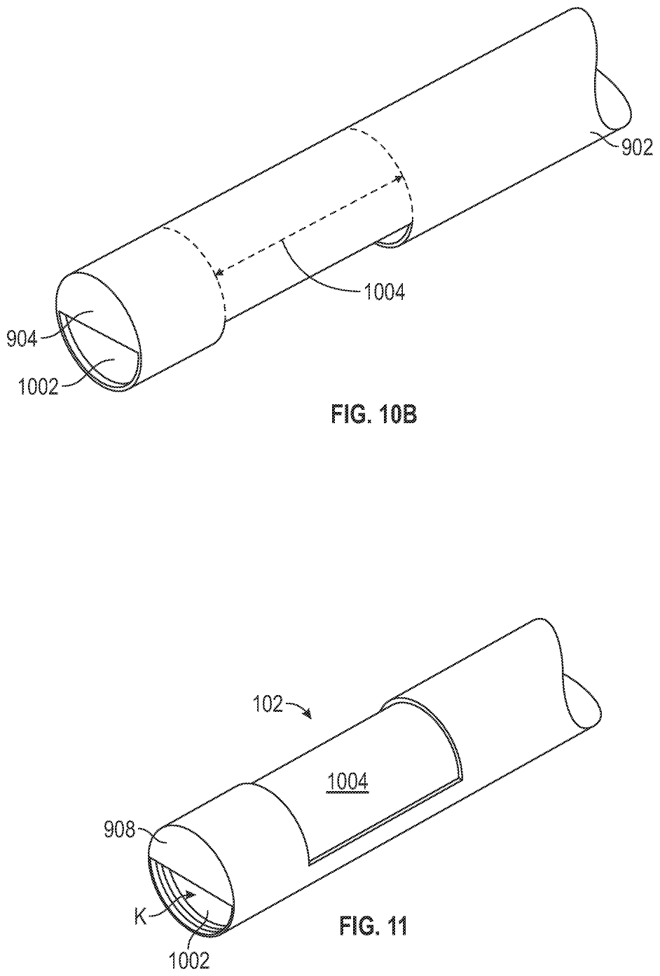

The resection assembly 102 can also have the configuration of FIGS. 9-11. Here, the resection assembly includes an outer tube 900 along with an inner tube 902 rotatably disposed within the outer tube 900. The inner tube 902 can rotate along the directions A and B into a first position shown in FIG. 9 and a second position shown in FIG. 11. In the first position, an end cap 904 of the inner tube 902 can align with a distal opening 906 of the outer tube 900 such that fluid can only enter the resection 102 through the resection element side window 302 as indicated by arrow Z. In the first position, a combination of an end cap 908 of the outer tube 900 and the inner tube end cap 904 can prevent entry of fluid at the distal end of the resection assembly 102.

Making reference to FIGS. 10A and 10B, the inner tube 902 can include a window 1000, which can align with the resection element side window 302 in the first position of FIG. 9 in order to allow entry of fluid as shown with arrow Z. Moreover, the inner tube 902 can include an end cap window 1002, which can align with the outer tube distal opening 906 in the second position of FIG. 11. Additionally, the inner tube 902 can include a blocking element 1004 which can align with resection element side window 302 in the second position of FIG. 11. Thus, when the inner tube 902 is rotated into the second position within the outer tube 900 in FIG. 11, the inner tube blocking element 1004 functions to close the resection element side window 302 such that fluid does not enter the resection element side window 302. Instead, fluid will enter the resection assembly 102 at the distal end as shown by arrow K through the outer tube distal opening 906 and the inner tube end cap window 1002, thereby allowing passage of fluid therethrough.

In the first position, tissue resection can occur in one of three ways. First, tissue resection can occur when the resection element 202 oscillates, as discussed above. Second, the resection element 202 can reciprocate along the directions X and Y. Third, the resection element 202 can oscillate along the directions A and B while simultaneously reciprocating along the directions X and Y. In the second position, an active electrode portion in conjunction with the return electrode can function to resect tissue at the target site. By virtue of using bipolar energy for resection, coagulation can simultaneously occur with resection.

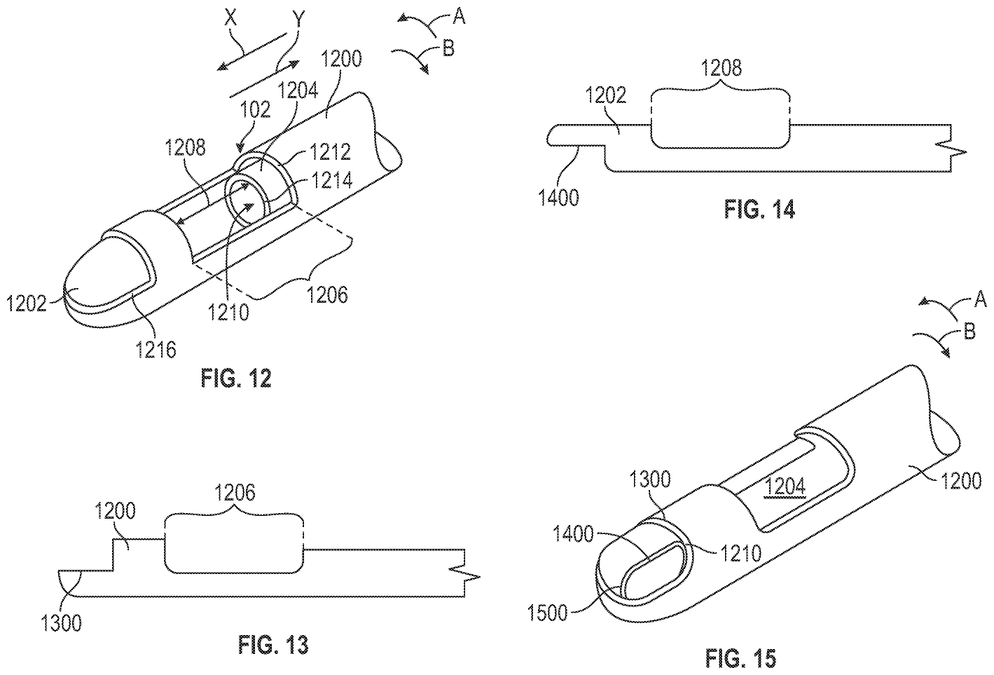

Moreover, the resection assembly 102 can have the configuration shown with reference to FIGS. 12-15. The resection assembly 102 can include an elongate member 1200 along with an oscillating resection element 1202 and a reciprocating resection element 1204. The elongate member 1200 can form a return electrode while each of the oscillating resection element 1202 and the reciprocating resection element 1204 can form an active electrode. Each of the elongate member 1200, the oscillating resection element 1202, the reciprocating resection element 1204 can be formed of the same materials as the elongate member 106. The oscillating resection element 1202 can be located within a lumen of the elongate member 1200 similar to the elongate lumen 200 and rotate along the directions A and B relative to the elongate member 1200. The reciprocating resection element 1204 can be located within a lumen of the oscillating resection element 1202 and slide along the directions X and Y relative to the oscillating resection element 1202. While not shown, an insulator having properties similar to the inner tube 204, can be disposed between the elongate member 1200 and the oscillating resection member 1202. Likewise, an insulator having properties similar to the inner tube 204 can be disposed between the oscillating resection member 1202 and the reciprocating resection element 1204.

The elongate member 1200 can include a side window 1206 and the oscillating resection element 1202 can include a side window 1208. In a first mode of operation where the elongate member 1200 and the oscillating resection element 1202 are in a first position, the elongate member side window 1206 can align with the oscillating resection element side window 1208 and provide a passage 1210 through which resected tissue from a target site such as the debris 504 can pass.

The elongate member 1200 can include a return electrode face 1212 and the reciprocating resection element 1204 can include an active electrode face 1214. In the first mode of operation, the reciprocating resection element active electrode face 1214 can be activated. Also, in the first mode of operation, the reciprocating resection element 1204 can move along the directions X and Y in order to resect tissue at a target site. When the reciprocating resection element active electrode face 1214 comes into contact with the target site, tissue can be resected from the target site and suction applied at the lateral suction port 108 can pull the resected tissue into the morcellator 100 via the passage 1210.

The elongate member 1200 can include an opening 1300 where a return electrode face 1216 can be disposed around the elongate member opening 1300. The oscillating resection element 1202 can include an opening 1400 where an active electrode 1500 can be disposed around the oscillating resection element opening 1400. In a second mode of operation, the reciprocating resection element 1204 can move into a second position as shown in FIG. 15 where the reciprocating resection element 1204 can substantially cover the elongate member side window 1206 and the oscillating resection element side window 1208. The reciprocating resection element 1204 can be coupled to the handle 120 and can move into the second position when a user moves the handle 120 along the direction A, thereby covering the elongate member side window 1206 and the oscillating resection element side window 1208. In the second mode of operation, the oscillating resection element opening 1400 can oscillate along the directions A and B within the elongate member opening 1300. In the second mode of operation, the morcellator 100 can be moved towards a backwall of a uterus. When the oscillating resection element opening 1400 comes into contact with a target site at the backwall, tissue can be resected from the target site and suction applied at the lateral suction port 108 can pull the resected tissue into the morcellator 100 via the oscillating resection element opening 1400 when the morcellator 100 operates in a bipolar mode.

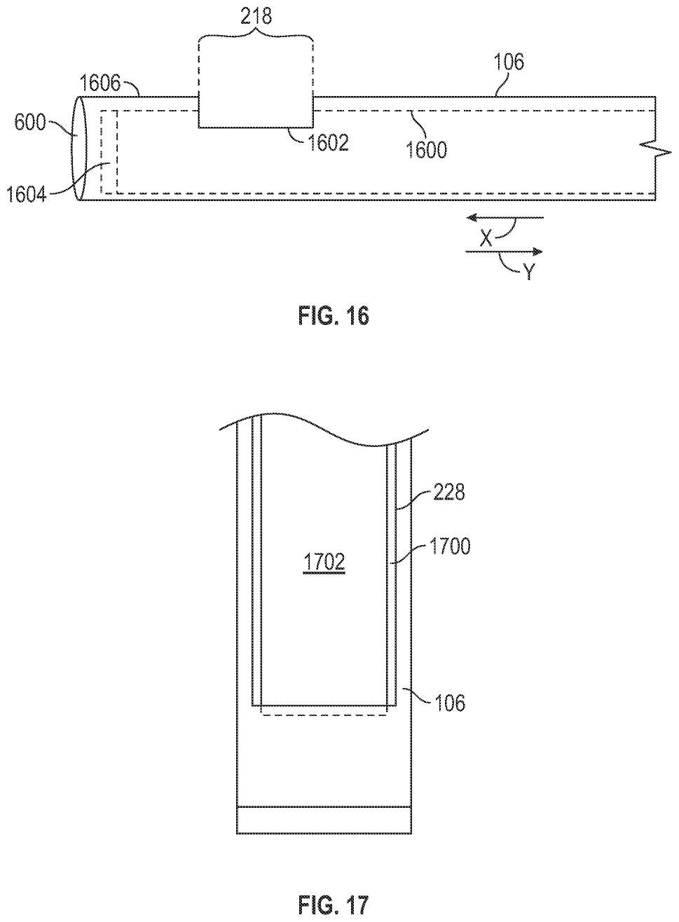

In addition to resecting tissue using bipolar energy, the morcellator 100 can implement mechanical features to resect tissue from a target site. For example, the morcellator 100 can include a resection element 1600 disposed within the lumen 200 of the elongate member 106, as shown in FIG. 16. The resection element 1600 can include a side window 1602 that can align with the elongate member side window 218.

The resection element 1600 can include a cutting edge 1700 that, during a first mode of operation, can resect tissue from a target site. The cutting edge 1700 can include a blade or any other type of mechanical cutting device suitable for resecting tissue from a target site. The cutting edge 1700 can oscillate relative to the elongate member 106 and the elongate member window 218 as discussed above in Figures SA and SB and the inner tube 204 relative to the cutting edge 220. During the first mode of operation, tissue, such as the debris 504, resected from a target site can flow into a passage 1702 via suction applied at the lateral suction port 108. The passage 1702 can be fluidly coupled with the lateral suction port 108 such that resected tissue can be pulled into the passage 1702 during the first mode of operation.

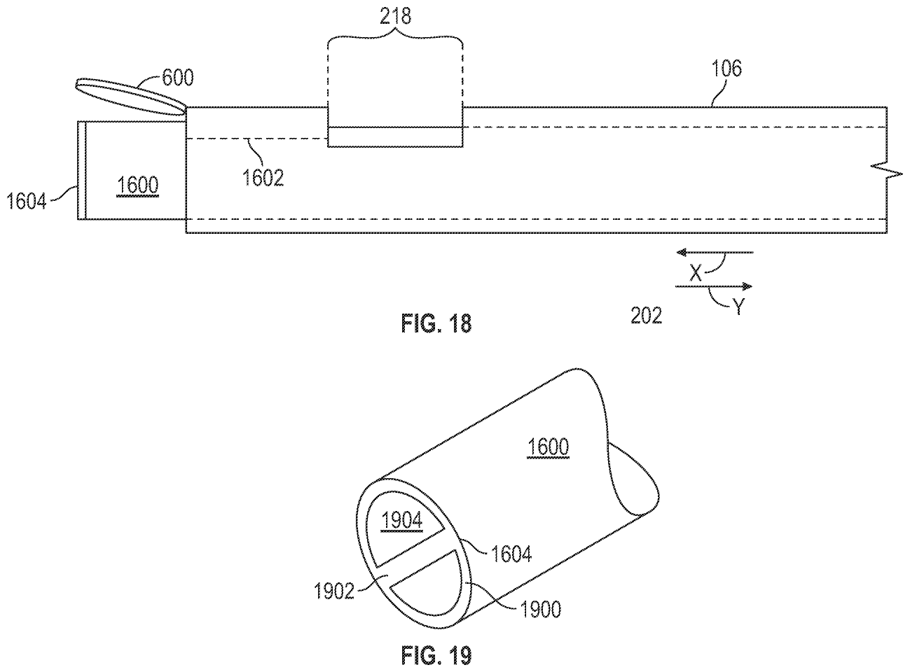

The resection element 1600 can also have a blade 1604 located at a distal end 1606 of the resection element 1600. In the example of FIG. 16, the morcellator 100 can include the end cap 600 that covers the resection element blade 1604. The resection element 1600 can be coupled to the handle 120 such that during a second mode of operation, a user can move the resection element 1600 along the direction X, thereby opening the end cap 600 and exposing the resection blade 1604, as shown in FIG. 18. The resection element blade 1604 can include a cutting edge 1900 along with a cross-member portion 1902. Each of the cutting edge 1900 and the cross-member portion 1902 can have the same characteristics as the cutting edge 220 discussed above.

In the second mode of operation, the cutting edge 1902 and the cross-member portion 1902 can be brought into contact with a target site such as a backwall of a uterus. The resecting element 1600 can be actuated, such as by a user rotating the handle 120, in order to resect tissue from the target site. A passage 1904 at the blade 1604 can be fluidly coupled with the lateral suction port 108. Thus, suction applied at the lateral suction port 108 can pull tissue resected from the target site with the resection element blade 1604 from the target site.

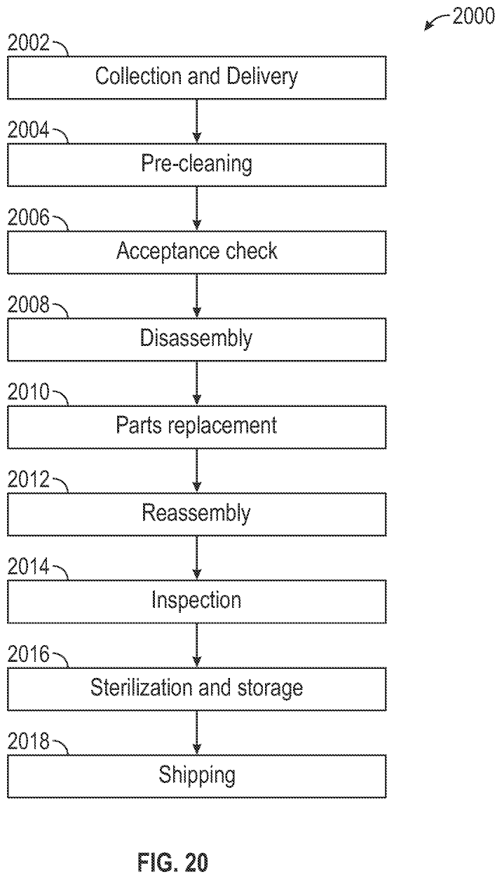

The morcellator 100 can be disposed of after a single use. Alternatively, the morcellator 100 can be repeatedly used a plurality of times. When the morcellator 100 can be repeatedly used a plurality of times, the morcellator 100 can be subjected to a method 2000, as shown in FIG. 20, which is a flowchart illustrating a reprocessing method for the hysteroscope 100. The morcellator 100 described above may be disposed of after one use or may be repeatedly used a plurality of times. When repeatedly used a plurality of times, the reprocessing method in FIG. 20 can be used. An operator who remanufactures devices can collect the used morcellator 100 after it has been used for treatment and can transport the morcellator 100 to a factory or other facility at 2002. The used morcellator 100 can be transported in a dedicated container such as to help prevent contamination from other treatments.

At 2004, the operator cleans and sterilizes the used morcellator 100. During cleaning, deposits adhering to the portions of the morcellator 100 are removed by using a brush or the like. Any cleaning solution of isopropanol-containing cleaning agent, proteolytic enzyme detergent, and alcohol can be applied to the morcellator 100 in order to remove pathogenic microorganisms and the like derived from blood, body fluid, or the like. The cleaning agent is not limited to the cleaning liquid described above, and other cleaning agents can be used. During sterilization, high-pressure steam sterilization, ethylene oxide gas sterilization, gamma ray sterilization, hydrogen peroxide and hydrogen peroxide low temperature sterilization can be applied to the morcellator 100. By virtue of the hysteroscope 100 having the structures described above, the hysteroscope 100 is easy to clean.

After cleaning and sterilization, an acceptance check of the hysteroscope 100 can be performed at 2006. During the acceptance check, an inspection of the hysteroscope 100 can be performed to determine if the morcellator 100 has any significant defects. Moreover, a number of times the morcellator 100 has been reprocessed can be determined. The number of times the morcellator 100 has been reprocessed can be compared against a threshold to determine if the number of times the hysteroscope 100 has been reprocessed exceeds a threshold and should no longer be used.

Next, at 2008, the hysteroscope 100 can be disassembled where various components of the morcellator 100, such as the resection assembly 102 or the elongate member 106 are removed from the morcellator 100. While only the resection assembly 102 and the elongate member 106 are referenced as being removed, any portion of the hysteroscope 100 described herein can be removed at 2008.

After disassembly at 2008, any components of the morcellator 100 that are deemed defective can be replaced at 2010. To further illustrate, if the resection assembly 102 is deemed defective, the resection assembly can be replaced at 2010. While the resection assembly 102 is mentioned as being replaced, any portion of the morcellator 100 described herein can be replaced at 2010.

After components of the morcellator 100 are replaced at 2010, the morcellator 100 can be reassembled at 2012. During reassembly, an identifier that can indicate the morcellator 100 has been modified from its original condition to include the replacement component(s) can be added. The identifier can include a label or any other type of indicia that designates the morcellator 100 as reprocessed, refurbished, or remanufactured.

The morcellator 100 is then inspected and tested at 2014. Specifically, a user can verify that the newly formed morcellator 100 has the same effectiveness and safety as the original product by various functional tests After inspection, the morcellator 100 can be sterilized and stored at 1016. The morcellator 100 can be sterilized with a sterilizing gas such as ethylene oxide gas or propylene oxide gas. After sterilization, the morcellator 100 can be stored at 2016 and then subsequently shipped at 2018.

Having described various aspects and features of the inventive subject matter, the following numbered examples are provided as illustrative embodiments:

Example 1 is a medical device comprising: an elongate member defining a lumen, the elongate member including: a side window at a distal portion of the elongate member; and a front window at a distal end of the elongate member, the front window defining an opening at the elongate member distal end; and a resection element extending through the elongate member lumen to the elongate member distal end, the resection element being movable within the elongate member lumen, the resection element having a side window that is alignable with the elongate member side window, the resection element includes, a cutting element at the resection element distal end.

In Example 2, the subject matter of Example 1 includes, wherein the cutting element is an electrically active electrode and the elongate member defines a return electrode.

In Example 3, the subject matter of Example 2 includes,) that extends across the resection element opening.

In Example 4, the subject matter of Examples 2-3 includes, wherein a portion of the cross-member forms a portion of the active element.

In Example 5, the subject matter of Examples 2-4 includes, wherein the resection element is rotatable and slidable within the elongate member lumen.

In Example 6, the subject matter of Examples 1-5 includes, wherein the front window is movable between an open position and a closed position and wherein the resection element is exposed at the front end when the front window is in the open position.

In Example 7, the subject matter of Example 6 includes, wherein the side window is in a closed position when the front window is an open position and the side window is in an open position when front window is in a closed position.

In Example 8, the subject matter of Examples 1-7 includes, wherein the resection element is slidable within the elongate member lumen between a first position relative to the elongate member lumen and a second position relative to the elongate member lumen.

In Example 9, the subject matter of Example 8 includes, wherein the elongate member includes an end cap that covers the front window.

In Example 10, the subject matter of Example 9 includes, wherein the end cap is coupled with the elongate member and moves between a closed position and an open position, wherein the end cap is in the closed position when the resection element is in the first position and the end cap is in the open position when the resection element is in the second position.

In Example 11, the subject matter of Examples 1-10 includes, wherein the resection element is rotatable within the elongate member lumen between a first position relative to the elongate member lumen and a second position relative to the elongate member lumen.

In Example 12, the subject matter of Example 11 includes, wherein the elongate member includes an end cap that partially covers the elongate member distal end such that a first portion of the elongate member distal end is covered with the end cap and a second portion of the elongate member includes the opening defined by the front window.

In Example 13, the subject matter of Example 12 includes, wherein a first portion of the resection electrode includes the active electrode that is covered by the end cap when the resection element is in the first position and active electrode aligns with the opening when the resection element is in the second position.

In Example 14, the subject matter of Examples 1-13 includes, wherein the resection element side window defines the active electrode.

In Example 15, the subject matter of Examples 1-14 includes, an insulator disposed between the elongate member and the resection element, the insulator defining an opening at the front window and a side window that is alignable with the elongate member side window and the resection element side window.

In Example 16, the subject matter of Examples 1-15 includes, wherein the resection element defines an inner tube disposed within the elongate member lumen and the medical device further comprises a mid-tube disposed between the inner tube and the elongate member.

In Example 17, the subject matter of Examples 1-16 includes, wherein the resection element is extendible distally outward from the front window.

Example 18 is a method for processing an instrument for surgery, the method comprising: sterilizing a medical device comprising: an elongate member defining a lumen, the elongate member including: a side window at a distal portion of the elongate member; and a front window at a distal end of the elongate member, the front window defining an opening at the elongate member distal end; and a resection element extending through the elongate member lumen to the elongate member distal end wherein a distal end of the resection element is exposed at the front window, the resection element being movable within the elongate member lumen, the resection element having a side window that is alignable with the elongate member side window, wherein the resection element includes, a cutting element at the resection element distal end; and storing the medical device in a sterile container.

Example 19 is at least one machine-readable medium including instructions that, when executed by processing circuitry, cause the processing circuitry to perform operations to implement of any of Examples 1-18.

Example 20 is an apparatus comprising means to implement of any of Examples 1-18.

Example 21 is a system to implement of any of Examples 1-18.

Example 22 is a method to implement of any of Examples 1-18.

The above detailed description includes references to the accompanying drawings, which form a part of the detailed description. The drawings show, by way of illustration, specific examples in which the invention can be practiced. These examples are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other examples can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed example. Thus, the following claims are hereby incorporated into the Detailed Description as examples or examples, with each claim standing on its own as a separate example, and it is contemplated that such examples can be combined with each other in various combinations or permutations. The scope of the invention should be determined with reference to the appended claims, along with the full scope of equivalents to which such claims are entitled.

What is claimed:

1. A medical device comprising:
an elongate member defining a lumen, the elongate member including:
a side window at a distal portion of the elongate member; and
a front window at a distal end of the elongate member, the front window defining an opening at the elongate member distal end; and
a resection element extending through the elongate member lumen to the elongate member distal end, the resection element being movable within the elongate member lumen, the resection element having a side window that is alignable with the elongate member side window, the resection element includes a cutting element at the resection element distal end.

2. The medical device of claim 1, wherein the cutting element is an electrically active electrode and the elongate member defines a return electrode.

3. The medical device of claim 2, wherein the resection element defines an opening at the elongate member front window and the resection element includes a cross-member that extends across the resection element opening.

4. The medical device of claim 2, wherein a portion of the cross-member forms a portion of the active element.

5. The medical device of claim 2, wherein the resection element is rotatable and slidable within the elongate member lumen.

6. The medical device of claim 1, wherein the front window is movable between an open position and a closed position and wherein the resection element is exposed at the front end when the front window is in the open position.

7. The medical device of claim 6, wherein the side window is in a closed position when the front window is an open position and the side window is in an open position when front window is in a closed position.

8. The medical device of claim 1, wherein the resection element is slidable within the elongate member lumen between a first position relative to the elongate member lumen and a second position relative to the elongate member lumen.

9. The medical device of claim 8, wherein the elongate member includes an end cap that covers the front window.

10. The medical device of claim 9, wherein the end cap is coupled with the elongate member and moves between a closed position and an open position, wherein the end cap is in the closed position when the resection element is in the first position and the end cap is in the open position when the resection element is in the second position.

11. The medical device of claim 1, wherein the resection element is rotatable within the elongate member lumen between a first position relative to the elongate member lumen and a second position relative to the elongate member lumen.

12. The medical device of claim 11, wherein the elongate member includes an end cap that partially covers the elongate member distal end such that a first portion of the elongate member distal end is covered with the end cap and a second portion of the elongate member includes the opening defined by the front window.

13. The medical device of claim 12, wherein a first portion of the resection electrode includes the active electrode that is covered by the end cap when the resection element is in the first position and active electrode aligns with the opening when the resection element is in the second position.

14. The medical device of claim 1, wherein the resection element side window defines the active electrode.

15. The medical device of claim 1, further comprising an insulator disposed between the elongate member and the resection element, the insulator defining an opening at the front window and a side window that is alignable with the elongate member side window and the resection element side window.

16. The medical device of claim 1, wherein the resection element defines an inner tube disposed within the elongate member lumen and the medical device further comprises a mid-tube disposed between the inner tube and the elongate member.

17. The medical device of claim 1, wherein the resection element is extendible distally outward from the front window.

18. A method for processing an instrument for surgery, the method comprising:
sterilizing a medical device comprising:
an elongate member defining a lumen, the elongate member including:
a side window at a distal portion of the elongate member; and
a front window at a distal end of the elongate member, the front window defining an opening at the elongate member distal end; and
a resection element extending through the elongate member lumen to the elongate member distal end wherein a distal end of the resection element is exposed at the front window, the resection element being movable within the elongate member lumen, the resection element having a side window that is alignable with the elongate member side window, wherein the resection element includes a cutting element at the resection element distal end; and
storing the medical device in a sterile container.
